(19) United States
(12) Patent Application Publication
(10) Pub. No.: US 2023/0178204 A1
(43) Pub. Date: Jun. 8, 2023

(54) SYSTEM AND METHOD TO IDENTIFY SUITABLE PATIENT SUBGROUPS FOR BIOLOGICS

(71) Applicant: Reciprocal Labs Corporation, Madison, WI (US)

(72) Inventors: Christopher HOGG, Madison, WI (US); Joseph SLAVINSKY, III, Madison, WI (US); Meredith Ann BARRETT, Madison, WI (US); Rahul Bhailal GONDALIA, Madison, WI (US); Leanne KAYE, Madison, WI (US)

(21) Appl. No.: 17/997,500

(22) PCT Filed: Apr. 30, 2021
(86) PCT No.: PCT/US2021/030170

§ 371 Date: Oct. 28, 2022

Related U.S. Application Data
(60) Provisional application No. 63/018,695, filed on May 1, 2020.

(51) Int. Cl. G16H 20/13; G16H 40/67; A61B 5/00

(52) U.S. Cl. CPC G16H 20/13; G16H 40/67; A61B 5/4839; A61B 5/4848; G09B 19/003

(57) ABSTRACT

A system and method to determine whether a patient should receive enhanced treatment of a respiratory ailment such as the prescription of biologics. Use data of a respiration medicament device to deliver controller or rescue respiration medicament to the patient is collected. The use data is transmitted to a storage device. The use data is stored in the storage device. Based on the use data, the system determines whether the patient is over a first threshold level of adherence in use of the respiration medicament device. Based on the use data, the system determines whether the patient has a rescue respiration medicament use over a second threshold level. A notification of recommendation of the enhanced treatment is provided if the patient is over the first and the second threshold.

### CROSS-REFERENCE TO RELATED APPLICATIONS

The application claims priority to, and benefit of, U.S. Provisional Patent Application Ser. No. 63/018,695 filed on May 1, 2020, which is hereby incorporated by reference herein in its entirety.

### TECHNICAL FIELD

The present disclosure relates generally to methods of improving treatment for patients with potential respiratory ailments, and more specifically to determining appropriate patient subgroups for biologics treatment.

### BACKGROUND

Respiratory ailments such as asthma remain a significant and costly public health problem. For example, in the United States, more than 22 million people have asthma. Worldwide, the World Health Organization estimates the population with asthma may be 235 million, and predicts that it will rise in the future. Similarly, recent studies by the Centers for Disease Control and Prevention have listed COPD as the third leading cause of death in the United States while estimating close to 15 million people may have COPD induced impaired lung function (Wheaton, A. G., Cunningham, T. J., Ford, E. S., Croft, J. B., Employment and activity limitations among adults with chronic obstructive pulmonary disease—United States, 2013. MMWR Morb. Mortal Wkly. Rep., 2015, 64(11), 290-295).

Despite the development of new medications, rates of hospitalizations and emergency room visits have not declined. Each year in the United States asthma causes approximately 2 million emergency department visits, 500,000 hospitalizations, and 5,000 deaths. In addition, asthma is responsible for an estimated 5.2 million missed days of school, and 8.7 million days of work (Nurmagambetov, T., Kuwahara, R., Garbe, P., The Economic Burden of Asthma in the United States, 2008-2013., Ann. Am. Thorac. Soc., 2018, 15(3), 348-356). Total annual costs to US health insurers and employers are greater than US$82 billion (ibid). COPD causes approximately 715,000 hospitalizations, and 134,000 deaths yearly. Additionally, the cost to the nation for COPD was recently projected to be approximately US$49.9 billion, including US$29.5 billion in direct health care expenditures, US$8.0 billion in indirect morbidity costs and US$12.4 billion in indirect mortality costs.

Many asthma exacerbations could be prevented with currently available treatments, however, only 1 in 5 asthmatics has the disease under control. Such treatments often rely on identifying a triggering condition of an asthma condition and properly administering treatment such as a medicament. One mechanism for a patient to self-administer a medicament is an inhaler. When a trigger event occurs, a patient may administer the medicament via a puff from the inhaler.

Revised national guidelines urge doctors to more closely monitor whether prescribed treatment for asthma is controlling everyday symptoms and improving quality of life. Healthcare providers, however, are limited by an availability of tools to assess how well their patients are doing on a day-to-day basis. An increasing number of physicians have begun to use periodic, written questionnaires (e.g. the asthma control test (ACT)) to monitor patients and determine their level of control. These instruments require patients to accurately recall and report the frequency of symptoms, medicament device (e.g. inhaler) usage, and activity level and restriction over some period of time, usually two to four weeks. As a result, collected information relating to management of respiratory disorders has been subject to error introduced by biases (e.g. fallible recollection), different interpretations of symptoms, and behaviors (e.g. non-adherence), and extremely limited frequency of data collection such that the data is of limited utility.

There are rare instances where standard rescue inhaler medicaments may be ineffective for certain respiratory ailments. One exciting new treatment is the use of biologics for treatment of severe respiratory ailments specifically asthma in the respiratory space. A biologic is a pharmaceutical drug product manufactured in, extracted from, or semi-synthesized from biological sources. The term “biologic” is used herein to the refer to any biologic-based treatment or therapy, thereby encompassing any biologic medical product or pharmaceutical drug. The challenge with prescription of biologics is that they are expensive and require either self-administration or a visit to a care provider for the treatment to be administered. It is also difficult to assess what type of effective biologic may be administered as it depends on numerous factors such as blood and sputum eosinophil count. Thus, healthcare actors are challenged in administering the biologics to the correct patient population.

There is a need for a system that allows for efficient selection of a group of patients for administration of biologics. There is a further need for a system that allows collection of data based on asthma controller and rescue medicament use to identify and support the authorization of a subgroup of patients for efficient biologic application. There is also a need for monitoring symptoms via rescue medication use and adherence to controller medications while a patient is administered biologics.

### SUMMARY

One disclosed example is a system to determine suitability for enhanced treatment for a respiratory ailment. The system includes a communication interface to collect use data of a respiration medicament device to deliver controller or rescue respiration medicament to a patient. The system includes a storage device to store the collected use data. A data analysis module is operable to determine whether the patient is over a first threshold level of adherence in use of the respiration medicament device based on the collected use data. The module determines whether the patient has a rescue respiration medicament use over a second threshold level based on the collected use data. The module provides a notification of recommendation of the enhanced treatment if the patient is over the first threshold and the second threshold.

A further implementation of the example system is where the respiratory ailment is asthma. Another implementation is where the enhanced treatment is prescription of a biologic-based treatment or therapy. Another implementation is where the first threshold is a percentage adherence value of at least about 65% adherence, such as at least about 70%, 75% 80% or 85% adherence. In one implementation, the first threshold is a percentage adherence value of at least about 75% adherence. Another implementation is where the second threshold is at least about fifteen uses of rescue medicament over one week, such as at least about twenty-two or twenty-eight uses of rescue medicament over one week. In one implementation, the second threshold is at least about twenty-eight uses of rescue medicament over one week. Another implementation is where the system includes a mobile computing device and the mobile computing device is in communication with the storage device and the communication interface. Another implementation is where the mobile computing device includes an application to assist the patient in applying the enhanced treatment. Another implementation is where the notification is provided electronically to one of the patient, a caregiver, or a healthcare provider. Another implementation is where the system includes an enhanced treatment module engine in communication with the mobile computing device. The enhanced treatment module is operable to track a use of the enhanced treatment by the patient. Another implementation is where the enhanced treatment module is operable to order additional enhanced treatment for the patient. Another implementation is where the enhanced treatment is prescription of an injectable biologic. The application includes an interface to coach the patient on at least one injection technique. Another implementation is where the interface includes an interface to record the area of an injection of the biologic. Another implementation is where the system includes a health monitor to monitor the patient. The data analysis module collects data from the health monitor to determine the response of the patient to the enhanced treatment.

Another disclosed example is a method of determining whether a patient should receive enhanced treatment of a respiratory ailment. Use data of a respiration medicament device to deliver controller or rescue respiration medicament to the patient is collected via a communication interface. The use data is transmitted to a storage device. The use data is stored in the storage device accessible to a data analysis module. It is determined whether the patient is over a first threshold level of adherence in use of the respiration medicament device based on the collected use data. It is determined whether the patient has a rescue respiration medicament use over a second threshold level based on the collected use data. A notification of recommendation of the enhanced treatment is provided if the patient is over the first threshold and the second threshold.

A further implementation of the example method is where the respiratory ailment is asthma. Another implementation is where the enhanced treatment is prescription of a biologic-based treatment or therapy. Another implementation is where the first threshold is a percentage adherence value of at least about 65% adherence, such as at least about 70%, 75% 80% or 85% adherence. In one implementation, the first threshold is a percentage adherence value of at least about 75% adherence. Another implementation is where the second threshold is at least about fifteen uses of rescue medicament over one week, such as at least about twenty-two or twenty-eight uses of rescue medicament over one week. In one implementation, the second threshold is at least about twenty-eight uses of rescue medicament over one week. Another implementation is where the method includes establishing communication to a mobile computing device with the storage device and the communication interface. Another implementation is where the mobile computing device includes an application to assist the patient in applying the enhanced treatment. Another implementation is where the notification is provided electronically to one of the patient, a caregiver, or a healthcare provider. Another implementation is where the method includes tracking a use of the enhanced treatment by the patient. Another implementation is where an enhanced treatment module is operable to order additional enhanced treatment for the patient. Another implementation is where the enhanced treatment is prescription of an injectable biologic. The application includes an interface to coach the patient on at least one injection technique. Another implementation is where the interface includes an interface to record the area of an injection of the biologic. Another implementation is where the method includes monitoring the patient via a health monitor to monitor the patient. The method also includes collecting data from the health monitor to determine the response of the patient to the enhanced treatment.

The above summary is not intended to represent each embodiment or every aspect of the present disclosure. Rather, the foregoing summary merely provides an example of some of the novel aspects and features set forth herein. The above features and advantages, and other features and advantages of the present disclosure, will be readily apparent from the following detailed description of representative embodiments and modes for carrying out the present invention, when taken in connection with the accompanying drawings and the appended claims.

### BRIEF DESCRIPTION OF THE DRAWINGS

The disclosure will be better understood from the following description of exemplary embodiments together with reference to the accompanying drawings, in which:

FIG. 1 shows a respiratory ailment analytics system for monitoring accurate, real-time medicament device usage, performing analytics on that data, and providing analysis of patients that may be subjects for enhanced treatment;

FIG. 2 is a high-level block diagram illustrating an example of a computing device used in either as a client device, application server, and/or database server;

FIG. 3 is a graph of patient groups for determination of a subgroup for biologics;

FIG. 4 shows screen images of example forms that may be created from the collected data for authorization for enhanced treatment;

FIG. 5 shows screen images generated by an example application for a patient to report injections of biologics;

FIG. 6A shows a screen image of a patient population outcome graph that may be used to demonstrate outcomes from a given patient population using electronic medication monitors;

FIG. 6B is a table of adherence correlated with rescue puffs for a patient population using electronic medication monitors; and

FIG. 7 is a flow diagram of a process of selection of a patient subgroup based on collection of medicament data.

The present disclosure is susceptible to various modifications and alternative forms. Some representative embodiments have been shown by way of example in the drawings and will be described in detail herein. It should be understood, however, that the invention is not intended to be limited to the particular forms disclosed. Rather, the disclosure is to cover all modifications, equivalents, and alternatives falling within the spirit and scope of the invention as defined by the appended claims.

### DETAILED DESCRIPTION OF THE ILLUSTRATED EMBODIMENTS

The present inventions can be embodied in many different forms. Representative embodiments are shown in the drawings, and will herein be described in detail. The present disclosure is an example or illustration of the principles of the present disclosure, and is not intended to limit the broad aspects of the disclosure to the embodiments illustrated. To that extent, elements and limitations that are disclosed, for example, in the Abstract, Summary, and Detailed Description sections, but not explicitly set forth in the claims, should not be incorporated into the claims, singly or collectively, by implication, inference, or otherwise. For purposes of the present detailed description, unless specifically disclaimed, the singular includes the plural and vice versa; and the word “including” means “including without limitation.” Moreover, words of approximation, such as “about,” “almost,” “substantially,” “approximately,” and the like, can be used herein to mean “at,” “near,” or “nearly at,” or “within 3-5% of,” or “within acceptable manufacturing tolerances,” or any logical combination thereof, for example.

The present disclosure relates to a system that collects data relating to individual-level patterns of rescue inhaler use and controller medication adherence. The data may be used to identify and describe subgroups of patients with respiratory ailments such as asthma for potential intervention using biologics treatment. Such subgroups of patients include those who require further treatment beyond rescue medicaments and those who are most likely to adhere to the prescribed biologics treatment.

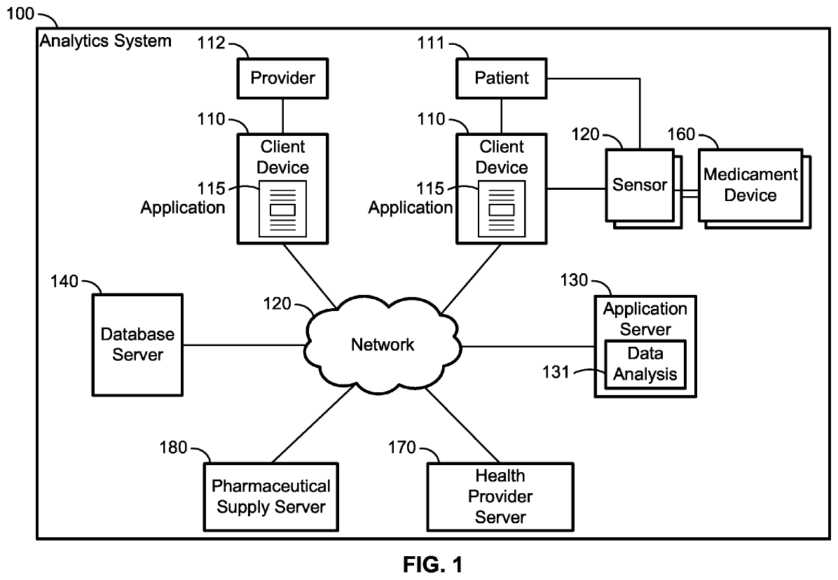

FIG. 1 shows a respiratory ailment analytics system 100 for monitoring accurate, real-time medicament device events, performing analytics on that data, and providing respiratory ailment exacerbation risk notifications based on identification of triggers for a primary patient in relation to a general patient population, according to one embodiment. Such respiratory ailments may be an asthma event that may be addressed through prompt treatment of medicament through an inhaler.

The respiratory ailment analytics system includes client computing devices 110, a medicament device sensor 120, a medicament device 160, an application server 130, database server 140, and a network 150. Other servers such as a health provider server 170 and a pharmaceutical supply server 180 may be coupled to the network 150. Although FIG. 1 illustrates only a single instance of most of the components of the respiratory ailment analytics system 100, in practice more than one of each component may be present, and additional or fewer components may be used.

The client devices 110, at the behest of their users, interact with the respiratory ailment analytics system 100 via the network 150. For purposes of explanation and clarity it is useful to identify at least two different types of users. A patient 111 is a user burdened with a respiratory ailment such as asthma who makes use of the respiratory ailment analytics system 100 at least in part to obtain personalized rescue event risk notifications provided by the server 130 and management notifications created by their healthcare provider 112 in this example. The patient 111 may be supported by a caregiver who is an additional or alternative user. The caregiver may have their own application 115 and client device 110 which receives the same notifications as the patient 111 (not shown in FIG. 1), or receives notifications on the patient's behalf (e.g. parents/guardians of patients 111 who may also want to receive notifications in the event that their own client devices 110 are distinct from that of their children). Such notifications can be provided in exchange for the user's permission to allow the respiratory ailment analytics system 100 to monitor the patient's 111 medicament device 160 usage. As will be explained below, medication events are detected by a sensor 120 associated with the medicament device 160 and the user's client device 110, which in turn reports to the application server 130, which in turn can initiate a process to generate risk notifications which are provided to the user through the client device 110. In this example, the patients 111 represent a patient population, for which individual data is taken for each patient in the group.

Another type of user is a healthcare provider 112 who, again with the express permission of the patient 111, also receives notifications regarding a patient's asthma management, as well as aggregated asthma community rescue event data and derived statistics regarding asthma events and other associated data. Other types of users may be contemplated.

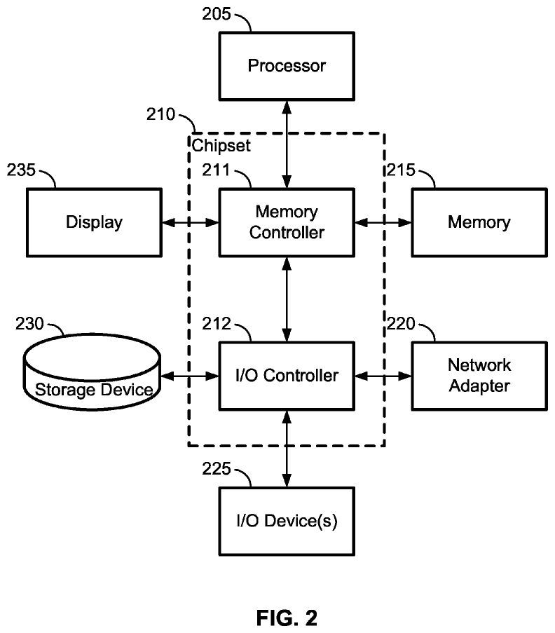

The client device 110 is a computer system that may be a portable computing device such as a smart phone, tablet, or laptop. The client device 110 could also be a television (e.g. a smart, connected television) or a speaker system (e.g. a smart, connected speaker). An example physical implementation is described more completely below with respect to FIG. 2. The client device 110 is configured to wirelessly communicate with the respiratory ailment analytics system 100 via the network 150. With network 150 access, the client device 110 transmits the time of medication usage events to system 100, as well as information describing the event as received from the associated medicament device sensor 120 (referred to throughout as “sensor 120”). The user's geographical location may also be transmitted to the respiratory ailment analytics system 100.

Regarding user location and event times, the client device 110 may determine the geographical location and time of a rescue event through use of information about the cellular or wireless network 150 to which it is connected. For example, the current geographical location of the client device 110 may be determined by directly querying the software stack providing the network 150 connection. Alternatively, the geographical location information may be obtained by pinging an external web service (not shown in FIG. 1) made accessible via network 150. The time of an event can be provided by the sensor 120 as part of the event data or added to event data by querying an appropriate software routine available as part of the client device's native operating system.

In addition to communicating with the application server 130, client devices 110 connected wirelessly to the respiratory ailment analytics system 100 may also exchange information with other connected client devices 110. For example, through a client software application 115, a healthcare provider 112 or caregiver may receive a risk exacerbation notification describing a recent rescue event about a patient 111, then in response send a recommendation to the patient 111 for post-asthma rescue event treatment. Similarly, through application 115 patients 111 may communicate with their healthcare providers 112 and other patients 111 or caregivers.

Application 115 provides a user interface (herein referred to as a “dashboard”) that is displayed on a screen of the client device 110 and allows a user to input commands to control the operation of the application 115. The dashboard is the mechanism by which healthcare providers 112, caregivers and patients 111 access the respiratory ailment analytics system 100. For example, the dashboard allows patients 111, caregivers and providers 112 to interact with each other, receive asthma rescue event risk notifications, exchange messages about treatment, provide and receive additional event and non-event data, and so on. Application 115 may be coded as a web page, series of web pages, or content otherwise coded to render within an internet browser. Application 115 may also be coded as a proprietary application configured to operate on the native operating system of the client device 110.

In addition to providing the dashboard, application 115 may also perform some data processing on asthma rescue event data locally using the resources of client device 110 before sending the processed data through the network 150. Event data sent through the network 150 is received by the application server 130 where it is analyzed and processed for storage and retrieval in conjunction with database server 140. The application server 130 may direct retrieval and storage request to the database server 140 as required by the client application 115. As will be explained, this analysis may be extended to include event data from multiple patients 111.

The client device 110 communicates with the sensor 120 using a network adapter and either a wired or wireless communication protocol, an example of which is the Bluetooth Low Energy (BTLE) protocol. BTLE is a short-ranged, low-powered, protocol standard that transmits data wirelessly over radio links in short range wireless networks. After the sensor 120 and client device 110 have been paired with each other using a BTLE passkey, the sensor 120 automatically synchronizes and communicates information relating to medicament device usage with the client device 110. If the sensor 120 hasn't been paired with a client device 110 prior to a rescue medication event, the information is stored locally until such a pairing occurs. Upon pairing, the sensor 120 communicates any stored event records to the client device 110. In other implementations, other types of wireless connections are used (e.g., infrared or IEEE 802.11).

Although client devices 110 and medicament devices 160 are described above as being separate physical devices (such as smart phones and inhalers, respectively), in the future it is contemplated the medicament devices 160 may include not only sensors 120 integrated into a single housing with the medicament device 160, but also aspects of the client device 110. For example, a medicament device 160 may include an audiovisual interface including a display or other lighting elements as well as speakers for presenting visual audible information. In such an implementation the medicament device 160 itself may present the contents of notifications provided by the server 130 directly, in place of or in addition to presenting them through the client devices 110.

The medicament device 160 is a medical device used to deliver medication to the lungs of a user experiencing a respiratory ailment rescue event. Different medicaments are used for asthma. Different medicaments may also be used for rescue and control for asthma. Medicament devices (e.g., inhalers) are typically portable and small enough to be carried by hand for ease of accessibility when treating respiratory attacks. In one embodiment, medication is delivered in aerosol form through a medicament device 160 such as a metered dose inhaler. Metered dose inhalers included a pressured, propellant canister of aerosol medication, a metering valve for delivering a regulated medicine dosage amount, and a plastic holder that holds the pressurized canister and also forms a mouthpiece for delivery of the medication. In another embodiment, medication is delivered in dry powder form through a medicament device 160 such as a dry powder inhaler. Dry powder inhalers may have Cartesian ovular shaped bodies that house wheel and gear mechanisms enabling a user to index through a strip of dry powder medication. The bodies of dry powder inhalers also include a manifold and a mouthpiece to deliver dry powder to the user. Examples of controller medications that are dispensed by a controller medicament device 160 include Beclomethasone Dipropionate, Beclomethasone Dipropionate HFA Budesonide, Ciclesonide, Flunisolide, Fluticasone Furoate, Fluticasone Propionate, Mometasone, Mometasone furoate HFA 100 or 200 mcg, and Triamcinolone Acetonideas, as well as combinations of those medications with a long-acting bronchodilator such as salmeterol or formoterol. Other medications may include long-acting beta-agonists (LABA) such as Albuterol Sulfate, Formoterol Fumarate, Salmeterol Xinafoate, Arformoterol Tartrate, and Olodaterol. The long-acting beta-agonists may also include combinations such as Budesonide in combination with Formoterol; Fluticasone furoate, umeclidinium and vilanterol; Fluticasone Propionate and Salmeterol; Fluticasone furoate 100 mcg and Vilanterol 25 mcg; Glycopyrrolate/Formoterol Fumarate; Indacaterol/glycopyrrolate; Mometasone in combination with Formoterol; Tiotropium 2.5 mcg and Olodaterol 2.5 mcg; and Umeclidinium and Vilanterol. Examples of rescue medications that are dispensed by a rescue medicament device 160 include Albuterol Sulfate, Albuterol Sulfate HFA, Albuterol Sulfate inhalation and nebulizer solution, salbutamol, Levalbuterol HCI, metaproterenol, and terbutaline.

Each patient may be associated with more than one medicament device 160. For example, the patient may have a rescue medicament device 160 that dispenses rescue medication, and a controller medicament device 160 that dispenses controller medication. Similarly, each patient may be associated with more than one sensor 120, each chosen to operate with one of the patient's medicament devices 160.

Generally, a sensor 120 is a physical device that monitors the usage of the medicament device 160. The sensor 120 is either removably attachable to the medicament device without impeding the operation of the medication device, or the sensor 120 is an integrated component that is a native part of the medicament device 160 as made available by its manufacturer.

The sensor 120 includes its own network adapter (not shown) that communicates with the client device 110 either through a wired connection, or more typically through a wireless radio frequency connection. In one embodiment, the network adapter is a Bluetooth Low Energy (BTLE) wireless transmitter, however in other embodiments other types of wireless communication may be used (e.g., infrared, IEEE 802.11).

The sensor 120 may also be configured to communicate more directly with the application server 130 to transmit collected data. For example, if the network adapter of the sensor 120 is configured to communicate via a wireless standard such as IEEE 802.11 or LTE, the adapter may exchange data with a wireless access point such as a wireless router, which may in turn communicate with the application server 130 without necessarily involving the client device 110 in every exchange of data. These two methods of communicating are not mutually exclusive, and the sensor 120 may be configured to communicate with both the client device 110 and the application server 130, for example using redundant transmission to ensure event data arrives at the application server 130 or to provide information directly to the client device 110 while the application server 130 is determining what notification to provide in response to an event.

As introduced above, the sensor 120 captures data about usage of the medicament device 160. Specifically, each sensor 120 captures the time and geographical location of the rescue medication event, that is, usages of the rescue medicament device 160, by the patient 111. Each sensor 120 transmits the event data in real-time or as soon as a network connection is achieved, automatically without input from the patient 111, caregiver or healthcare provider 112. The medication event information is transmitted to the application server 130 for use in analysis, generation of asthma rescue event notifications, and in aggregate analyses of event data across multiple patients.

To accomplish this goal, there are a number of different ways for the sensor 120 to be constructed, and in part the construction will depend upon the construction of the medicament device itself 160. Generally, all sensors 120 will include an onboard processor, persistent memory, and the network adapter described above, that together, function to record, store, and report medication event information to the client device 110 and/or server 130. Sensors 120 may also include a clock for recording the time and date of events.

Regarding specific constructions of the sensor 120, traditional inhalers, such as mechanical dose counters, are not designed with sensors 120 in mind, and thus the sensor 120 may be constructed accordingly. Some implementations in this manner include mechanical, electrical, or optical sensors to detect movement of the device 160, priming of the device, activation of the device, inhalation by the user, etc. In contrast, modern inhalers, such as deflectable membrane dose counters, include electrical circuitry may report event information as an electrical data signal which a sensor 120 is designed to receive and interpret, for example the medicament device 160 itself may report movement, priming, and activation to the sensor 120.

More information regarding hardware and software components for the sensors 120 and medicament devices 160, as well as the interaction between them to record rescue medication events can be found in U.S. patent application Ser. No. 12/348,424, filed Jan. 1, 2009, and International Application No. PCT/US2014/039014, filed May 21, 2014, both of which are incorporated by reference herein in their entirety.

The application server 130 is a computer or network of computers. Although a simplified example is illustrated in FIG. 2, typically the application server will be a server class system that uses powerful processors, large memory, and faster network components compared to a typical computing system used, for example, as a client device 110. The server typically has large secondary storage, for example, using a RAID (redundant array of independent disks) array and/or by establishing a relationship with an independent content delivery network (CDN) contracted to store, exchange and transmit data such as the asthma notifications contemplated above. Additionally, the computing system includes an operating system, for example, a UNIX operating system, LINUX operating system, or a WINDOWS operating system. The operating system manages the hardware and software resources of the application server 130 and also provides various services, for example, process management, input/output of data, management of peripheral devices, and so on. The operating system provides various functions for managing files stored on a device, for example, creating a new file, moving or copying files, transferring files to a remote system, and so on.

The application server 130 includes a software architecture for supporting access and use of the analytics system 100 by many different client devices 110 through network 150, and thus at a high level can be generally characterized as a cloud-based system. The application server 130 generally provides a platform for patients 111 and healthcare providers 112 to report data recorded by the sensors associated with their medicament devices 160 including both rescue medication and controller medication events, collaborate on respiratory ailment treatment plans, browse and obtain information relating to their condition and geographic location, and make use of a variety of other functions.

Generally, the application server 130 is designed to handle a wide variety of data. The application server 130 includes logical routines that perform a variety of functions including checking the validity of the incoming data, parsing and formatting the data if necessary, passing the processed data to a database server 140 for storage, and confirming that the database server 140 has been updated.

The application server 130 stores and manages data at least in part on a patient by patient basis. Towards this end, the application server 130 creates a patient profile for each user. The patient profile is a set of data that characterizes a patient 111 of the respiration ailment analytics system 100. The patient profile may include identity information about the patient such as age, gender, current rescue medication, current controller medication, notification preferences, a controller medication adherence plan, a relevant medical history, and a list of non-patient users authorized to access to the patient profile. The profile may further specify a device identifier, such as a unique media access control (MAC) address identifying the one or more client devices 110 or sensors 120 authorized to submit data (such as controller and rescue medication events) for the patient.

The profile may specify which different types of notifications are provided to patients 111 or caregivers and their personal healthcare providers 112, as well as the frequency with which notifications are provided. For example, a patient 111 or caregiver may authorize a healthcare provider 112 to receive notifications indicating a rescue event. The patient 111 or caregiver may also authorize their healthcare provider 112 be given access to their patient profile and rescue event history. If the healthcare provider 112 is provided access to the patient profile of the patient 111, the healthcare provider may specify controller adherence or rescue medication plans for the corresponding asthma condition. Medication plans may include a prescribed number of doses per day for controller medications.

The application server 130 also creates profiles for healthcare providers 112. A healthcare provider profile may include identifying information about the healthcare provider 112, such as the office location, qualifications and certifications, and so on. The healthcare provider profile also includes information about their patient population. The provider profile may include access to all of the profiles of that provider's patients, as well as derived data from those profiles such as aggregate demographic information, rescue and controller medication event patterns, and so on. This data may be further subdivided according to any type of data stored in the patient profiles, such as by geographic area (e.g., neighborhood, city) over by time period (e.g., weekly, monthly, or yearly).

The application server 130 receives rescue medication event information from the client device 110 or the sensor 120, triggering a variety of routines on the application server 130. In the example implementations described below, the data analysis module 131 executes routines to access respiratory ailment event data as well as other data including a patient's profile, analyze the data, and output the results of its analysis to both patients 111 or caregivers and providers 112. This process is generally referred to as a respiratory ailment risk analysis. In this example, the risk analysis is performed in relation to asthma for the patient 111. The asthma risk analysis may be performed at any point in time, in response to a rescue event, due to a relevant change in the patient's environment, and in response to any one of a number of triggering conditions discussed further below.

Other analyses are also possible. For example, a risk analysis may be performed on rescue and controller medication use for multiple patients to identify based on spatial/temporal clusters (or outbreaks) of medication use based on historically significant permutations from individual, geographic, clinical, epidemiologic, demographic, or spatial or temporal baselines or predicted or expected values. Other types of analyses may include daily/weekly adherence trends, adherence changes over time, adherence comparisons to other relevant populations (e.g., all patients, patients on a particular rescue medication or controller medication or combination thereof, identification of triggers (spatial, temporal, environmental), rescue use trends over time, and rescue use comparisons to other relevant populations. Thus, the percentage of adherence for a particular patient is calculated by the number of controller puffs that are taken on a specific day divided by the daily number of puffs that are prescribed. This daily metric is then averaged over a specified time period to get the mean daily adherence. The number of controller puffs prescribed is based on the patients' medication plan, which is entered into the application.

Responsive to any analyses performed, the application server 130 prepares and delivers push notifications to send to patients 111, authorized healthcare providers 112, and/or other users provided access to the patient's profile, such as caregivers. Notifications can provide details about the timing, location, and affected patient(s) 111 involved in a medication rescue event. Notifications may additionally comprise a distress or emergency signal that requests emergency assistance that are distributed to emergency assistance providers 112. Notifications may also include the results of the asthma risk analysis performed by the data analysis module 131. More information regarding the types of notifications that may be sent and the content they may contain is further described below.

In addition to providing push notifications in response to an asthma risk analysis, notifications may also be provided as pull notifications, at particular time intervals. Additionally, some notifications (whether push or pull) may be triggered not in response to an asthma risk analysis performed in response to a rescue medication event, but instead in response to a risk analysis performed in response to one of the underlying factors in the asthma risk analysis changing, such that an updated notification is warranted. For example, if weather conditions indicate that an increase in air pollution is occurring or is imminent, this may trigger conducting asthma risk analyses for all patients located in the particular geographic area where the pollution is occurring.

Notifications are provided through the network 150 to client applications 115 in a data format specifically designed for use with the client applications, and additionally or alternatively may be provided as short message service (SMS) messages, emails, phone calls, or in other data formats communicated using other communication mediums.

The database server 140 stores patient and provider data related data such as profiles, medication events, patient medical history (e.g., electronic medical records). Patient and provider data are encrypted for security and is at least password protected and otherwise secured to meet all Health Insurance Portability and Accountability Act (HIPAA) requirements. Any analyses (e.g., asthma risk analyses) that incorporate data from multiple patients (e.g., aggregate rescue medication event data) and are provided to users is de-identified so that personally identifying information is removed to protect patient privacy.

The database server 140 also stores non-patient data used in asthma risk analyses. This data includes regional data about a number of geographic regions such as public spaces in residential or commercial zones where patients are physically located and may be exposed to pollutants. This data may specifically include or be processed to obtain a patient's proximity to green space (areas including concentrated numbers of trees and plants). One example of regional data includes georeferenced weather data, such as temperature, wind patterns, humidity, the air quality index, and so on. Another example is georeferenced pollution data, including particulate counts for various pollutants at an instance of time or measured empirically. The regional data includes information about the current weather conditions for the time and place of the rescue event such as temperature, humidity, and air quality index. All of the items of data above may vary over time, and as such the data itself may be indexed by time, for example separate data points may be available by time of day (including by minute or hour), or over longer periods such as by day, week, month, or season. Although the database server 140 is illustrated in FIG. 1 as being an entity separate from the application server 130, the database server 140 may alternatively be a hardware component that is part of another server such as server 130, such that the database server 140 is implemented as one or more persistent storage devices, with the software application layer for interfacing with the stored data in the database is a part of that other server 130.

The database server 140 stores data according to defined database schemas. Typically, data storage schemas across different data sources vary significantly even when storing the same type of data including cloud application event logs and log metrics, due to implementation differences in the underlying database structure. The database server 140 may also store different types of data such as structured data, unstructured data, or semi-structured data. Data in the database server 140 may be associated with patients, groups of patients, and/or entities. The database server 140 provides support for database queries in a query language (e.g., SQL for relational databases, JSON NoSQL databases, etc.) for specifying instructions to manage database objects represented by the database server 140, read information from the database server 140, or write to the database server 140.

The network 150 represents the various wired and wireless communication pathways between the client devices 110, the sensor 120, the application server 130, and the database server 140. The network 150 uses standard Internet communications technologies and/or protocols. Thus, the network 150 can include links using technologies such as Ethernet, IEEE 802.11, integrated services digital network (ISDN), asynchronous transfer mode (ATM), etc. Similarly, the networking protocols used on the network 150 can include the transmission control protocol/Internet protocol (TCP/IP), the hypertext transport protocol (HTTP), the simple mail transfer protocol (SMTP), the file transfer protocol (FTP), etc. The data exchanged over the network 150 can be represented using technologies and/or formats including the hypertext markup language (HTML), the extensible markup language (XML), etc. In addition, all or some links can be encrypted using conventional encryption technologies such as the secure sockets layer (SSL), Secure HTTP (HTTPS) and/or virtual private networks (VPNs). In another embodiment, the entities can use custom and/or dedicated data communications technologies instead of, or in addition to, the ones described above.

FIG. 2 is a high-level block diagram illustrating physical components of an example computer 200 that may be used as part of a client device 110, application server 130, and/or database server 140 from FIG. 1, according to one embodiment. Illustrated is a chipset 210 coupled to at least one processor 205. Coupled to the chipset 210 is volatile memory 215, a network adapter 220, an input/output (I/O) device(s) 225, a storage device 230 representing a non-volatile memory, and a display 235. In one embodiment, the functionality of the chipset 210 is provided by a memory controller 211 and an I/O controller 212. In another embodiment, the memory 215 is coupled directly to the processor 205 instead of the chipset 210. In some embodiments, memory 215 includes high-speed random access memory (RAM), such as DRAM, SRAM, DDR RAM or other random access solid state memory devices.

The storage device 230 is any non-transitory computer-readable storage medium, such as a hard drive, compact disk read-only memory (CD-ROM), DVD, or a solid-state memory device. The memory 215 holds instructions and data used by the processor 205. The I/O device 225 may be a touch input surface (capacitive or otherwise), a mouse, track ball, or other type of pointing device, a keyboard, or another form of input device. The display 235 displays images and other information from the computer 200. The network adapter 220 couples the computer 200 to the network 150.

As is known in the art, a computer 200 can have different and/or other components than those shown in FIG. 2. In addition, the computer 200 can lack certain illustrated components. In one embodiment, a computer 200 acting as server 140 may lack a dedicated I/O device 225, and/or display 218. Moreover, the storage device 230 can be local and/or remote from the computer 200 (such as embodied within a storage area network (SAN)), and, in one embodiment, the storage device 230 is not a CD-ROM device or a DVD device.

Generally, the exact physical components used in a client device 110 will vary in size, power requirements, and performance from those used in the application server 130 and the database server 140. For example, client devices 110, which will often be home computers, tablet computers, laptop computers, or smart phones, will include relatively small storage capacities and processing power, but will include input devices and displays. These components are suitable for user input of data and receipt, display, and interaction with notifications provided by the application server 130. In contrast, the application server 130 may include many physically separate, locally networked computers each having a significant amount of processing power for carrying out the asthma risk analysis introduced above. In one embodiment, the processing power of the application server 130 provided by a service such as Amazon Web Services™. Also, in contrast, the database server 140 may include many, physically separate computers each having a significant amount of persistent storage capacity for storing the data associated with the application server.

As is known in the art, the computer 200 is adapted to execute computer program modules for providing functionality described herein. A module can be implemented in hardware, firmware, and/or software. In one embodiment, program modules are stored on the storage device 230, loaded into the memory 215, and executed by the processor 205.

In this example, an application executed on the application server 130 may collect data such as use data from medicament devices such as the device 160 in FIG. 1. The application collects data from uses of medicament devices by a patient population that can be used to qualify patients with moderate/severe respiratory ailments such as asthma who may be eligible for biologic treatments. Suitable biologic treatments may include for example omalizumab (Xolair), mepolizumab (Nucala), reslizumab (Cinqair), benralizumab (Fasenra), and dupilumab (Dupixent).

For example, collected data may be used to determine factors such as: a) use of inhaled corticosteroids (ICS) or bronchodilator medicine such as LABA; b) proof of use of IC S/LABA; and c) proof of controller adherence. Additional inhaled corticosteroids data relating to asthma control may be analyzed. Such data relating to asthma may include rescue inhaler usage, nighttime symptoms, an asthma control score (ACT), asthma control by ACT, and external sensor readings such as spirometry readings.

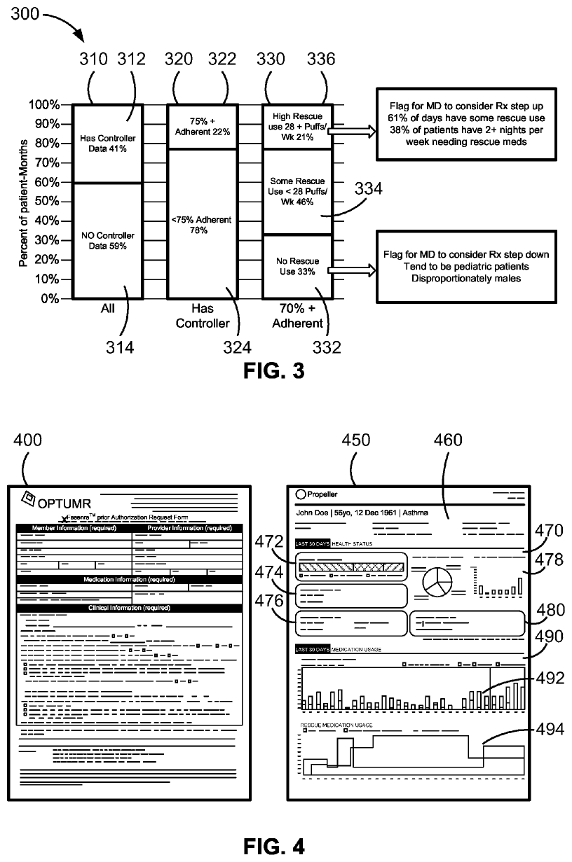

FIG. 3 is a graph 300 of breakdown of potential patients based on collected use data of medicament devices such as control and rescue inhalers. As explained herein, the use data may be collected by the analytics system 100 in FIG. 1. In this example, a first bar 310 shows all patients. The bar 310 is broken down into patients that have associated controller medicament data 312 and patients where controller medicament data is not available 314. A second bar 320 represents the selection of patients with controller medicament data 312. These patients are broken down into those patients that are over 75% adherent to their controller medicament instructions 322 and those patients that are under 75% adherent 324. Of course, other thresholds may be used to separate the patient population. For example, any suitable threshold value may be selected from about over 65% adherent. For example, a threshold value between 65% and 85% could be used. Such values may be at least about 65%, 70%, 80% or 85% adherence. In this example, 2 to 3 months (e.g., 60 or 90 days) of patient data is collected to determine the thresholds, but longer or shorter periods may be used.

A third bar 330 represents a first group of patients with no rescue events reported 332, a second group of patients with rescue events under a certain threshold such as under 28 puffs per week 334, a third group of patients with high rescue use 336. Other thresholds for rescue use may be used such as any threshold value over 15 uses of rescue medicament over one week. For example, such values may be in a range between 15-28 uses such as 15 or 22 uses of rescue medicament over one week. In this example, the first group of patients 332 and second group of patients 334 should continue the rescue medicament prescription therapy. The third group of patients 336 may be considered for recommendations of biologics treatment.

In this example, the system 100 in FIG. 1 may be used to notify users and their caregivers and clinicians via a health care system including the health provider server 370 that the patient may be an appropriate candidate for biologics based on the collected data explained above. As explained above, since the collected data is the same data that must be collected by a provider to justify the prescription of a biologics, the system may retain the collected data and make it available to candidate patients for biologics to streamline the application process. Such data may include proof of use of ICS/ long-acting beta-agonists (LABAs); proof of lack of disease control; exacerbation history; and proof of response/improvement to treatment.

FIG. 4 shows screen images of an example prior authorization form 400 and an example respiratory ailment report 450 that both may be generated from the patient and use data collected by the system 100. The prior authorization form 400 includes data that may be populated from the data collected by the system 100 to streamline the application process for enhanced treatment.

The respiratory ailment report 450 in this example relates to asthma and may be used to document the prescription of enhanced treatment. The respiratory ailment report 450 includes a patient details field 460, a health status field 470, and a medication usage field 490. The patient details field 460 includes information about the patient and the types of medication currently prescribed. The health status field 470 includes a control status graph 472, a night time rescue use data field 474, a controller adherence field 476, a rescue trend graphic 478, and an asthma control test result field 480. The control status graph 472 in this example provides a graph that shows the percentage of status of asthma as well controlled, not well controlled and poorly controlled over a pre-determined period of time such as 30 days. The night time rescue use data field 474 shows the number of nights over the pre-determined period of time that required rescue use of the inhaler by the patient. The controller adherence field 476 shows the percentage of adherence of using an inhaler or inhalers over the pre-determined period of time. The rescue trend graphic 478 shows different graphics showing the time of day of rescue usage and the day of the week of rescue usage. The asthma control test result field 480 shows the results of the asthma control test.

The medication usage field 490 includes a rescue medication usage graph 492 that shows the days during the pre-determined period rescue medication use occurred as well as the times during the night and the total amount of times during the particular day. The medication usage field 490 also includes a controller medication usage graph 494 that shows the adherence to different controller medications.

Once the biologics are prescribed, the system 100 may continue to monitor the use of such biologics via an application executed on the patient computing device 110 in FIG. 1. The system 100 may also provide reminders to patients based on a medication treatment plan, i.e. to encourage adherence. The system 100 may also provide helpful information such as providing a user interface that includes visual or written directions to entering an injection location and walks them through the steps of injection.

Such an application may include a dashboard interface that allows a patient to enter occurrences of taking the biologics as part of the enhanced treatment. The interface may also collect patient reported outcomes from the administration of the biologics. This data may be reported to the application server 130 along with prescription and dispensing information from the pharmaceutical supply server 180 to track the biologics for a particular patient. The application executed on the patient computing device 110 may be tailored to specific application of biologics and therefore provide biologic specific content to help users understand how to administer biologics, educational content to help users better understand the benefits of biologics, educational content to help users better understand their asthma.

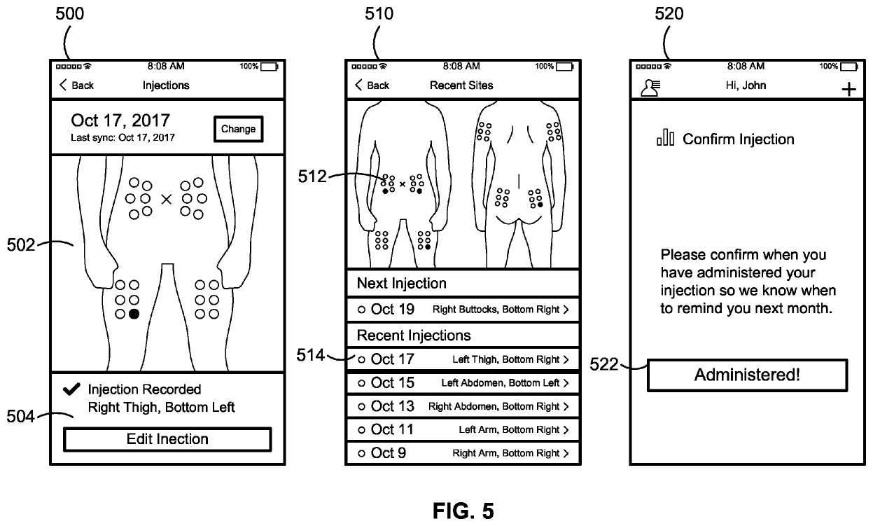

FIG. 5 shows screen images of interfaces generated by an example application executed on the patient computing device 110 to report injections of biologics. An injections interface 500 shows a location graph 502 that represents areas on a body graphic of where injections may occur. A patient selects the appropriate area, and a confirmation message 504 is displayed that indicates the injection is recorded and the location of the injection. A recent sites interface 510 shows a body graphic 512 that shows the locations of recent and projected future injections. The application may also display instructions and/or provide audio instructions as to how injections are to be made. A summary table 514 lists recent injections by date and body location as well as the next scheduled injection. A confirmation interface 520 provides a button 522 that allows a patient to confirm a reported injection has occurred. The application may include a reminder function that will remind the patient when to inject biologics and include features such as pop-up windows or a count-down clock. The application may include an interface to allow a user to select the biologics and set a schedule for injections of the biologics and corresponding reminders. The application may include trend graphics showing the historic record of previous injections. Such a historic record may be interposed with other patient data such as ACT history or inhaler doses. The application may allow a user to obtain further details such as the body location, time and medication of past injections. The application may also include interfaces with educational information on respiratory ailments and the treatments such as the particular biologic. The system 100 may receive the data from the application, track the injection sites, and make recommendations for future injections based on the historical data. The system 100 may also analyze data received in relation to historical trends from the application and infer events such as exacerbations.

The system 100 may be used to streamline re-ordering of biologic medications and provide reminders to re-order the medications. The system 100 can track shipping status to increase medication fulfillment and adherence. The use data as well as other patient data may be transmitted to a pharmaceutical distribution system that may include the pharmaceutical supply server 180. The tracking of the patient and the use of the biologics allows notifications to be sent to the patient, caregiver, healthcare provider, or other parties to predict re-ordering of the biologics.

The patient reported outcomes (PRO) and sensor data collected by the system 100 can be used to measure, and potentially predict responsiveness to a specific biologic. Examples of patient reported outcomes useful for asthma treatment may include blood eosinophil count, FeNO, allergen-driven symptoms, childhood-onset asthma, exacerbations in a prior time period, adult-onset asthma, nasal polyposis, and mild/severe atopic dermatitis. If data suggests a non-significant response to a prescribed biologic, the system could recommend changing to a different biologic after a given amount of time. The system 100 may also analyze/identify subgroups of patients who are the most responsive to different biologics. Responses to biologics could be based on inhaler use, and/or associated with other sensor data, e.g. respiratory rate, heart rate, bronchoconstriction, or biomarker in breath. Thus, data may be collected through a sensor that may include sensor reported rescue inhaler usage, sensor reported nighttime waking, and sensor-derived respiratory control. Night time awakenings and night time use of the rescue inhaler may be a proxy for waking up at night to relieve symptoms. Having nighttime awakenings is associated with poor outcomes in asthma. Through rescue use patterns (day and night) and the Asthma Control Test, asthma control specifically may be determined.

As explained above, the application may include an interface for self-reporting by the patient. Such data may include self-reported ACT data, self-reported quality of life, and self-reported exacerbations such as hospitalization, prescriptions of OCS, or appointments with healthcare providers. Other sensor data may be collected by the system 100 that may be included in the analysis of the responsiveness to a specific biologic. These may include passive activity, sleep data, at-home spirometry readings, and response to pollutants and irritants. Thus, night time awakenings may be characterized by night time inhaler use, or a sleep tracking sensor. For example, the system could be linked to sleep data from a wearable sensor, such as a FitBit, or a phone application such as the application available from Sleep Score Labs. Another source of data may include a data connection to a smart-syringe. Such smart syringes are known in the art, and could be connected to the current system to confirm use of the biologic, time and date of use, quantity of drug, batch of drug, etc.

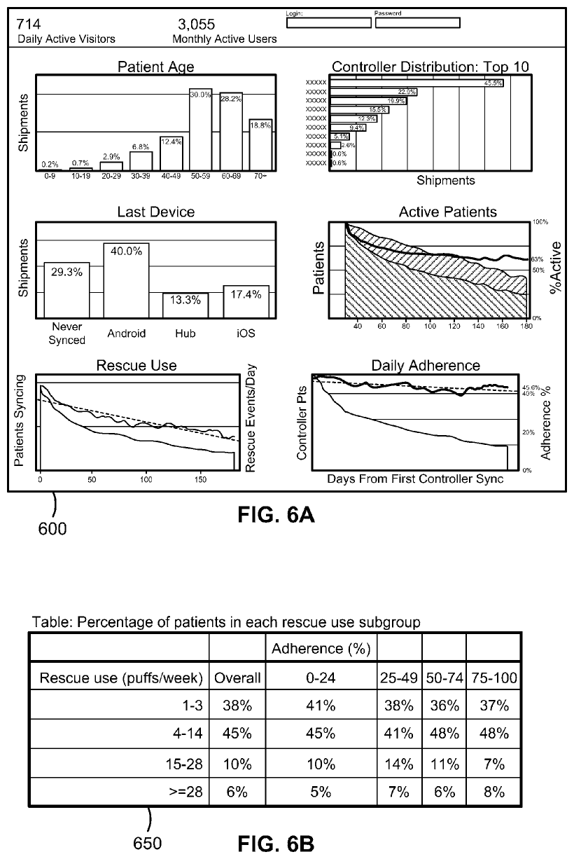

FIG. 6A shows an example screen image of a patient population outcome graph 600 that summarizes collected data for a given patient population. The graph 600 and the collected data may be used to determine the suitability of certain patients for biologics treatment, i.e. by viewing population-level characteristics and classifying the population into different adherence and rescue use classes that can be used to make clinical decisions in relation to biologics treatment (as in FIG. 6B), and/or analyze outcomes for the patient population that have been or are subject to biologics treatment.

In one example, patients over 18 years of age with self-reported asthma were enrolled in a digital health program over an eighteen month period. The program included electronic medication monitors (EMMs) that objectively and passively monitored inhaler use by the patients. The patients had both controller and rescue inhalers to treat their asthma. The use data generated by the inhalers was transmitted to dedicated smartphone applications associated with each patient. In this example, patients with greater than 90 days of EMM use were categorized into groups according to controller medication adherence and rescue use. FIG. 6B is a table 650 of adherence correlated with rescue puffs for the patient population. The adherence groups included quartiles of 0-24, 25-49, 50-74, and 75-100% adherence. The groups of rescue use include patients who applied 1-3 puffs, 4-14 puffs, 15-28 puffs, and greater than 28 puffs per week. Multivariable logistic regressions identified whether age, gender, Asthma Control Test (ACT) score (5-15, 16-19, >20), daily applications opens, and census tract-level demographics were associated with adherence and rescue use groups. The confidence level was a=0.05.

As shown in the table 650, the percentage of study patients (n=1263, mean age: 39 years, 79% female) with 75-100% adherence and greater than 28 rescue puffs/week and both was 18%, 6% and 1.5%, respectively. Models identified that older age, with an ACT score of greater than 20 and higher applications opens were associated with having 75-100% adherence, while those with an ACT score of 5-15 and higher application opens were associated with having greater than 28 rescue puffs/week.

The continuous monitoring in the system in FIG. 1 may be used for determining patients for selection of biologics to treat a variety of respiratory disorders other than asthma such as COPD, cystic fibrosis, and bronchiectasis. However, it is to be understood and appreciated that the principles described above is not to be limited to such use.

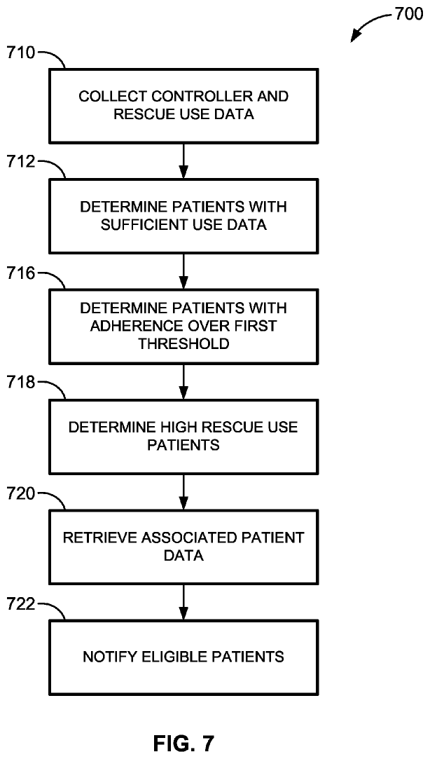

FIG. 7 is a flow diagram 700 of a data collection and analysis routine to determine patients for enhanced treatment such as biologics, according to certain aspects of the present disclosure. The flow diagram in FIG. 7 is representative of example machine readable instructions for the process of collecting data and selecting patients for enhanced treatment. In this example, the machine readable instructions comprise an algorithm for execution by: (a) a processor; (b) a controller; and/or (c) one or more other suitable processing device(s). The algorithm may be embodied in software stored on tangible media or computer program product such as flash memory, CD-ROM, floppy disk, hard drive, digital video (versatile) disk (DVD), or other memory devices having non-transitory computer readable media. However, persons of ordinary skill in the art will readily appreciate that the entire algorithm and/or parts thereof can alternatively be executed by a device other than a processor and/or embodied in firmware or dedicated hardware in a well-known manner (e.g., it may be implemented by an application specific integrated circuit [ASIC], a programmable logic device [PLD], a field programmable logic device [FPLD], a field programmable gate array [FPGA], discrete logic, etc.). For example, any or all of the components of the interfaces can be implemented by software, hardware, and/or firmware. Also, some or all of the machine readable instructions represented by the flowcharts may be implemented manually. Further, although the example algorithm is described with reference to the flowchart illustrated in FIG. 7, persons of ordinary skill in the art will readily appreciate that many other methods of implementing the example machine readable instructions may alternatively be used. For example, the order of execution of the blocks may be changed, and/or some of the blocks described may be changed, eliminated, or combined.

The routine first collects controller and rescue use data from a general population of patients that use respiratory medications (710). The use data is collected over a period of time such as 90 days. The routine identifies those patients for which there is sufficient data in relation to rescue and controller use (712). The routine then examines the patients for which there is sufficient data. The routine analyzes the use data and determines the patients for which adherence is over a first threshold value over a set period of time (714). In this example, the first threshold is patients that are over 75% adherent.

The routine then analyzes those patients that have an adherence over the first threshold value to determine those patients with a high rescue use (716). In this example, the definition of high rescue use is rescue use over a second threshold such as greater than 28 puffs a week. The routine then stores those patients that have a high rescue use (718). Any relevant medical data is retrieved for association with the patients (720). The routine then provides a notification that the patients in the last group are eligible for enhanced treatment such as biologics (722).

As used in this application, the terms “component,” “module,” “system,” or the like, generally refer to a computer-related entity, either hardware (e.g., a circuit), a combination of hardware and software, software, or an entity related to an operational machine with one or more specific functionalities. For example, a component may be, but is not limited to being, a process running on a processor (e.g., digital signal processor), a processor, an object, an executable, a thread of execution, a program, and/or a computer. By way of illustration, both an application running on a controller, as well as the controller, can be a component. One or more components may reside within a process and/or thread of execution, and a component may be localized on one computer and/or distributed between two or more computers. Further, a “device” can come in the form of specially designed hardware; generalized hardware made specialized by the execution of software thereon that enables the hardware to perform specific function; software stored on a computer-readable medium; or a combination thereof.

The terminology used herein is for the purpose of describing particular embodiments only, and is not intended to be limiting of the invention. As used herein, the singular forms “a,” “an,” and “the” are intended to include the plural forms as well, unless the context clearly indicates otherwise. Furthermore, to the extent that the terms “including,” “includes,” “having,” “has,” “with,” or variants thereof, are used in either the detailed description and/or the claims, such terms are intended to be inclusive in a manner similar to the term “comprising.”

Unless otherwise defined, all terms (including technical and scientific terms) used herein have the same meaning as commonly understood by one of ordinary skill in the art. Furthermore, terms, such as those defined in commonly used dictionaries, should be interpreted as having a meaning that is consistent with their meaning in the context of the relevant art, and will not be interpreted in an idealized or overly formal sense unless expressly so defined herein.

While various embodiments of the present invention have been described above, it should be understood that they have been presented by way of example only, and not limitation. Although the invention has been illustrated and described with respect to one or more implementations, equivalent alterations and modifications will occur or be known to others skilled in the art upon the reading and understanding of this specification and the annexed drawings. In addition, while a particular feature of the invention may have been disclosed with respect to only one of several implementations, such feature may be combined with one or more other features of the other implementations as may be desired and advantageous for any given or particular application. Thus, the breadth and scope of the present invention should not be limited by any of the above described embodiments. Rather, the scope of the invention should be defined in accordance with the following claims and their equivalents.

1. A system to determine suitability for enhanced treatment for a respiratory ailment, the system comprising:
a communication interface to collect use data of a respiration medicament device to deliver controller or rescue respiration medicament to a patient;
a storage device to store the collected use data; and
a data analysis module operable to:
based on the collected use data, determine whether the patient is over a first threshold level of adherence in use of the respiration medicament device;
based on the collected use data, determine whether the patient has a rescue respiration medicament use over a second threshold level; and
provide a notification of recommendation of the enhanced treatment if the patient is over the first threshold and the second threshold.

2. The system of claim 1, wherein the respiratory ailment is asthma.

3. The system of claim 1, wherein the enhanced treatment is prescription of a biologic-based treatment or therapy.

4. The system of claim 1, wherein the first threshold is a percentage adherence value of at least about 65% adherence.

5. The system of claim 1, wherein the second threshold is over at least about fifteen uses of rescue medicament over one week.

6. The system of claim 1, further comprising a mobile computing device, the mobile computing device in communication with the storage device and the communication interface, wherein the mobile computing device includes an application to assist the patient in applying the enhanced treatment.

7-8. (canceled)

9. The system of claim 6, further comprising an enhanced treatment module engine in communication with the mobile computing device, wherein the enhanced treatment module is operable to track a use of the enhanced treatment by the patient.

10. (canceled)

11. The system of claim 6, wherein the enhanced treatment is prescription of an injectable biologic, and wherein the application includes an interface to coach the patient on at least one injection technique.

12. The system of claim 11, wherein the interface includes an interface to record the area of an injection of the biologic.

13. The system of claim 1, further comprising a health monitor to monitor the patient, the data analysis module further operable to collect data from the health monitor to determine the response of the patient to the enhanced treatment.

14. A method of determining whether a patient should receive enhanced treatment of a respiratory ailment, the method comprising:
collecting use data of a respiration medicament device to deliver controller or rescue respiration medicament to the patient via a communication interface;
transmitting the use data to a storage device;
storing the use data in the storage device accessible to a data analysis module;
based on the collected use data, determining whether the patient is over a first threshold level of adherence in use of the respiration medicament device;
based on the collected use data, determining whether the patient has a rescue respiration medicament use over a second threshold level; and
providing a notification of recommendation of the enhanced treatment if the patient is over the first threshold and the second threshold.

15. The method of claim 14, wherein the respiratory ailment is asthma.

16. The method of claim 14, wherein the enhanced treatment is prescription of a biologic-based treatment or therapy.

17. The method of claim 14, wherein the first threshold is a percentage adherence value of at least about 65% adherence.

18. The method of claim 14, wherein the second threshold is at least about fifteen uses of rescue medicament over one week.

19. The method of claim 14, further comprising establishing communication to a mobile computing device with the storage device and the communication interface, wherein the mobile computing device includes an application to assist the patient in applying the enhanced treatment.

20-21. (canceled)

22. The method of claim 19, further comprising tracking a use of the enhanced treatment by the patient.

23. (canceled)

24. The method of claim 19, wherein the enhanced treatment is prescription of an injectable biologic, and wherein the application includes an interface to coach the patient on at least one injection technique and an interface to record the area of an injection of the biologic.

25. (canceled)

26. The method of claim 14, further comprising:
monitoring the patient via a health monitor to monitor the patient; and
collecting data from the health monitor to determine the response of the patient to the enhanced treatment.

27-28. (canceled)

29. A non-transitory computer readable medium having stored thereon software instructions that, when executed by a processor, cause the processor to:
collect use data of a respiration medicament device to deliver controller or rescue respiration medicament to the patient via a communication interface;
transmit the use data to a storage device;
store the use data in the storage device accessible to a data analysis module;
based on the collected use data, determine whether the patient is over a first threshold level of adherence in use of the respiration medicament device;
based on the collected use data, determine whether the patient has a rescue respiration medicament use over a second threshold level; and
provide a notification of recommendation of the enhanced treatment if the patient is over the first threshold and the second threshold.
